(19) United States
(12) Patent Application Publication
(10) Pub. No.: US 2005/0149050 A1
(43) Pub. Date: Jul. 7, 2005

(54) Arrangement and method for the intra-operative determination of the position of a joint replacement implant

(76) Inventors: Jan Stifter, Unterehrendingen (CH); Holger Broers, Uplengen (DE)

Correspondence Address: KNOBBE MARTENS OLSON & BEAR LLP, 2040 MAIN STREET, FOURTEENTH FLOOR, IRVINE, CA, 92614, US

(21) Appl. No.: 10/994,188

(22) Filed: Nov. 19, 2004

Related U.S. Application Data
(63) Continuation of application No. PCT/EP03/04469, filed on Apr. 29, 2003.

(30) Foreign Application Priority Data
May 21, 2002 (DE) 102 22 415.3
Feb. 18, 2003 (DE) 103 06 793.0

(51) Int. Cl. A61B 17/90; A61B 5/107

(52) U.S. Cl. 606/102; 606/87; 606/91; 600/587; 600/594

(57) ABSTRACT

Arrangement for the intra-operative determination of the spatial position and angular position of a joint replacement implant, especially a hip socket or shoulder socket or an associated stem implant, or a vertebral replacement implant, especially a lumbar or cervical vertebral implant, using a computer tomography method, having: a computer tomography modeling device for generating and storing a three-dimensional image of a joint region or vertebral region to be provided with the joint replacement implant, an optical coordinate-measuring arrangement for providing real position coordinates of defined real or virtual points of the joint region or vertebral region and/or position reference vectors between such points within the joint region or vertebral region or from those points to joint-function-relevant points on an extremity outside the joint region or vertebral region, the coordinate-measuring arrangement comprising a stereocamera or stereocamera arrangement for the spatial recording of transducer signals, at least one multipoint transducer, which comprises a group of measurement points rigidly connected to one another, and an evaluation unit for evaluating sets of measurement point coordinates supplied by the multipoint transducer(s) and recorded by the stereocamera, and a matching-processing unit for real position matching of the image to the actual current spatial position of the joint region or vertebral region with reference to the real position coordinates of the defined points, the matching-processing unit being configured for calculating transformation parameters with minimalization of the normal spacings.

### RELATED APPLICATIONS

This is a Continuation of PCT application PCT/EP03/04469, which was filed Apr. 29, 2003 and published in German on Nov. 27, 2003 as WO 03/096870, and which is incorporated herein by reference. The above PCT application claims priority to German patent application Serial Nos. 102 22 415.3, filed May 21, 2002 and 103 06 793.0, filed Feb. 18, 2003.

### BACKGROUND

1. Field of the Invention

The invention relates to an arrangement for the intra-operative determination of the position of a joint replacement implant, especially a hip socket or shoulder socket or an associated stem implant or a vertebral replacement implant, using a computer tomography method. It relates also to a corresponding method.

2. Description of the Related Art

Surgical interventions for the replacement of joints or joint components in human beings have been known for a long time and form part of everyday clinical procedure in industrialized countries. For decades, intensive development work has also been carried out with a view to the provision and continuing improvement of such implants, especially hip joint implants but increasingly also knee, shoulder and elbow joint implants as well as vertebral replacement implants. In parallel with those developments, which have now resulted in an almost infinite variety of such implant structures, there are also being made available and further developed suitable operating techniques and aids, including, especially, tools for the installation of implants that are matched to the implant structures in question.

It will also be understood that joint replacement operations are preceded by the acquisition of suitable images of the joint region in question, on the basis of which the operating surgeon determines a suitable implant and the surgical technique. Whereas formerly X-ray images were generally used for this purpose, in recent years computer tomograms have increasingly become the everyday tool of the operating surgeon. Nevertheless, the long-term success of joint replacement implantations is even today still closely associated with the experience of the operating surgeon, and this must to a considerable extent be attributed to the difficulties, which are not to be underestimated, of appropriate intra-operative utilization of visual images for achieving optimum alignment of the components of the joint implant in relation to the effective centers of rotation and load axes of the individual patient.

In recent years, therefore, there have been increased efforts to provide suitable positioning aids and methods for the operating surgeon, which have been derived substantially from developments in the field of robotics and manipulation techniques.

EP 0 553 266 B1 and U.S. Pat. No. 5,198,877 describe a method and an apparatus for contactless three-dimensional shape detection, which has provided stimulus for the development of medical “navigation” systems and methods; see also the detailed literature references in those specifications.

U.S. Pat. No. 5,871,018 and U.S. Pat. No. 5,682,886 disclose methods of ascertaining the load axis of the femur. In accordance with those methods, in a first step the coordinates of the femur are ascertained, for example by means of a computer tomography image, and stored in a computer. The stored data are then used to create a three-dimensional computer model of the femur and, with the aid of that model, the optimum coordinates are calculated for the positioning of a jig on the bone and of a knee prosthesis that is subsequently to be installed. The basis for this is the calculation of the load axis of the femur.

After such a simulation, the patient's femur is fixed in position and, using a registration device, contact is made with individual points on the femur surface in order to establish the orientation of the femur for the operation to be carried out. Such contacting of the bone requires either that the femur be exposed along large portions of its length, if possible as far as the hip joint, in order that its surface can be contacted with the registration device or that a kind of needle be used as a probe for penetrating through the skin as far as the bone. Since, however, any surgical intervention constitutes a risk to the patient and needle pricks cause bleeding and an additional risk of infection in the region of the bones, it is undesirable to perform an additional surgical intervention in the hip region or to insert needles along the femur in order to establish the location of the center of rotation. Furthermore, the femur needs to be firmly fixed on the measurement table of a registration device, because otherwise the hip socket may become displaced during the probing procedure, with the possibility that, once the registration of the femur coordinates is complete, the cutting jig will be incorrectly positioned.

FR 2 785 517 describes a method and a device for detecting the center of rotation of the head of the femur in the hip socket. For this purpose, the femur is moved with its head in the hip socket and the measurement point coordinates recorded in various positions of the femur are stored. As a soon as a shift in the center of rotation of the femur occurs, a corresponding counter-pressure is exerted on the head of the femur, which is taken into account in the determination of a point which relates to the arrangement of the femur.

DE 197 09 960 A1 describes a method and a device for the pre-operative determination of position data of endoprosthetic components of a central joint relative to the bones forming the central joint, it being proposed that an outer articulation point be determined by moving each of the bones about an outer joint located at the end of the bone in question that is remote from the central joint; that in the region of the said central joint an articulation point likewise be determined for each of the two bones; that by joining with a straight line the two articulation points so found for each of the two bones there be determined a direction characteristic thereof and finally that the orientation of the endoprosthetic components relative to that characteristic direction be determined.

Similar medical “navigation” methods are described in WO 95/00075 and WO 99/23956 wherein image-acquisition systems of the kind mentioned above are used for recording the position of references on the bones adjacent to the joint in question and characteristic points and axes can be derived from the virtual representation of the bone or joint obtained by that means.

A system of that kind, which has been improved in respect of reliability and, especially, in respect of independence from intra-operative movements of the patient and which is intended for direct use during surgery, is the subject of the Applicant's specification WO 02/17798 A1.

### SUMMARY

Starting from the prior art, the invention is based on the problem of providing an arrangement of that kind which is quickly and easily operated by the operating surgeon with a very low risk of error and which enables significantly improved surgical results to be achieved.

This problem is solved in terms of apparatus by an arrangement having the features of claim 1 and in terms of method by a method having the features of claim 15. The subsidiary claims relate to advantageous variants of the inventive concept. Their subject matter, in any combination with one another, including modifications, lie within the scope of the present invention.

The invention includes the basic concept of providing an integrated arrangement for the intra-operative determination of the spatial position and angular position of a joint replacement implant, which comprises essentially a computer tomography modeling device, an optical coordinate-measuring arrangement for providing real position coordinates of points or position reference vectors of a relatively narrow (or relatively wide) joint region that are relevant to the operation, and a matching-processing unit for the real position matching of the CT image. The invention also includes the concept of configuring the last-mentioned component of the system for the calculation of transformation parameters in accordance with the principle of the minimalization of the normal spacings.

In a preferred variant, the matching-processing unit is configured for carrying out an interactive adjustment procedure for matching a sensed bone surface to a corresponding virtual surface of the image with the combined application of the principle of triangular meshing and a spatial spline approach with the definition of the unknowns as spline parameters. This variant largely avoids the disadvantages associated with pure triangular meshing on the one hand and the spatial spline approach on the other hand, namely on the one hand the occurrence of jumps and edges in the generation of a surface of a 3D model and on the other hand excessive vibration in marginal regions. In the combined procedure favored here, it is specifically in marginal regions and poorly defined regions that the surface is generated using triangular meshing methods.

In a variant tailored to practical surgical use, the arrangement has an input interface for entering implant parameters of a predetermined set of suitable implants and for specifying possible implant positions and alignments in relation to the image, which interface is connected to the computer tomography modeling device and is especially in the form of an interactive user interface having means for user guidance. The matching-processing unit is connected to the input interface and is configured for determining desired coordinates or a desired movement vector of the implant being installed and a resection area or resectioning instrument therefor from at least one set of entered implantation parameters, positions and alignments. The input interface is configured especially for the inputting and image integration of the relevant body axis vectors and the implant parameters of a hip socket, especially the coordinates of the center of rotation as well as the anteversion angle and the abduction angle.

This variant therefore provides the operating surgeon with highly developed user guidance for the largely automated determination of a position and alignment of the joint replacement implant that is optimum in respect of the individual anatomical relationships. On the basis of previously obtained computer tomograms and a surgical plan developed on that basis, the surgeon can therefore make computer-based deductions as to the essential decisions to be taken intra-operatively, so that significantly higher accuracy is achieved and serious positioning errors can be virtually ruled out. Advantageously the optical coordinate-measuring arrangement comprises, in addition to the stereocamera or stereocamera arrangement, a first multipoint transducer which is in the form of a movable hand-guided sensor for sensing bony references in the joint region or vertebral region in order to determine the coordinates thereof. A second multipoint transducer is configured for rigid attachment to a bone or vertebra in the joint region or vertebral region, respectively.

With a view to providing an integrated total arrangement there is also included a resectioning instrument, especially a milling tool or a rasp, which can be rigidly connected to the second or a third multipoint transducer to form a geometrically calibrated, navigable tool/transducer unit. The transducer signals of that unit can be used to determine real position coordinates of an operational part of the resectioning instrument, especially a milling head or a rasp part, and therefrom, as desired, real position coordinates of a resection zone produced with the resectioning instrument. In that case the input interface is configured for entering instrument parameters of the resectioning instrument which allow its synoptic display with the image of the joint region or vertebral region obtained by the computer tomography modeling device. A further distinguishing feature of this variant is that the matching-processing unit is configured for allocating the real position coordinates of the operational part and, as desired, the real position coordinates of the resection zone to the image of the joint region or vertebral region substantially in real time. Finally, the arrangement comprises an image-display unit which is configured for synoptic display of the operational part or resection zone in its current position with the image of the joint region or vertebral region matched to real position coordinates.

In a further development of the inventive concept, the total arrangement also includes a mounting tool, especially a screwing tool, which can be rigidly connected to the second or third multipoint transducer to form a geometrically calibrated, navigable tool/transducer unit. The transducer signals of that unit can be used to determine real position coordinates of an operational part of the mounting tool and thus, as desired, of the implant itself. Further distinguishing features of this arrangement are that the input interface is configured for entering tool parameters of the mounting tool which allow its synoptic display with the image of the joint region or vertebral region obtained by the computer tomography modeling device and that the matching-processing unit is configured for allocating the real position coordinates of the operational part and, as desired, of the implant to the image of the joint region or vertebral region substantially in real time. In this case the image-display unit is then configured for synoptic display of the operational part or implant in its real position with the image of the joint region or vertebral region matched to real position coordinates.

In the above variants, the resectioning instrument and/or the mounting tool is in the form of a hand-guided tool with a handgrip having an attachment portion for rigid connection to the multipoint transducer. It will be understood that in the case of implant systems associated with a plurality of resectioning or mounting tools, the latter should advantageously all have a respective attachment portion in order to provide computer-based navigation suitable for all resectioning and mounting steps.

In a further advantageous development of the inventive concept, the total arrangement comprises an adapter component for the rigid attachment of a multipoint transducer to the joint replacement implant, especially at the proximal end of a stem implant, in order to create a navigable implant/transducer unit. The transducer signals of that unit can be used to determine real position coordinates of the adapter and thus, as desired, of the implant itself. Distinguishing features of this variant are that the input interface is configured for entering adapter parameters which allow synoptic display of the adapter or of the implant with the image of the joint region or vertebral region obtained by the computer tomography modeling device and that the matching-processing unit is configured for allocating the real position coordinates of the adapter and, as desired, of the implant to the image of the joint region or vertebral region substantially in real time. In this case—analogously to the variants mentioned above—the image-display unit is configured for synoptic display of the adapter or implant in its real position with the image of the joint region or vertebral region matched to real position coordinates.

The multipoint transducer(s) is(are) preferably in the form of passive four-point transducers having four spherical reflector parts. The stereocamera or camera arrangement is associated with an illuminating device with which the multipoint transducer(s) are illuminated, so that defined reflections for “imaging” the multipoint transducer in question are available. In order largely to exclude light that would disturb the operating surgeon, the illuminating device preferably operates in the infrared range.

In order to ensure the use of all relevant implant structures when an operation is carried out using the proposed arrangement, the user interface has a multi-region memory for storing the implant parameters of the suitable implants or a data bank interface to an implant parameter data bank. Furthermore, as already discussed above, the user interface has means for providing menu guidance, and these are here configured for carrying out an interactive process of selecting a component with repeated access to the multi-region memory or to the implant parameter data bank.

A variant of the proposed arrangement that provides especially extensive support for the operating surgeon comprises a control signal generation unit that is connected to the evaluation unit and to the matching-processing unit. This is configured for comparing a set of implant position data or alignment data that has been entered by means of the input interface and matched to the real position coordinates of the joint region or vertebral region with currently acquired real position coordinates of the operational part of the resectioning instrument or mounting tool or implant and for determining any variance between desired position and actual position coordinates and for outputting variance data or a control command derived from the variance, especially by means of a text or speech output and/or in a synoptic display with the image.

As regards the method aspects of the invention, they correspond substantially to the apparatus aspects discussed above, reference being made expressly thereto.

An advantageous procedure for carrying out the method comprises especially first entering implantation parameters of a predetermined set of suitable joint replacement implants or vertebral replacement implants and image-related desired coordinates for specifying possible implant positions and alignments thereof. The input is preferably effected by importing the data or implantation parameters of the relevant implants from a suitable database or—as regards the desired coordinates—in the context of a computer-based surgical plan, which has been organized especially in the form of interactive user guidance. Such a method also comprises the integration of an image of the joint replacement implant or vertebral replacement implant into the image of the body environment and the display of a synoptic representation from the images prior to the matching-processing step.

In a further development of the method, in which the navigation of a resectioning instrument or mounting tool or of the implant itself has been incorporated into the sequence, first of all the real position coordinates of an operational part of a resectioning instrument or mounting tool or of a joint replacement implant or vertebral replacement implant rigidly connected to a multipoint transducer are recorded by means of the coordinate-measuring arrangement. This is followed by the integration of the real position coordinates of a resection zone or the joint replacement implant or vertebral replacement implant ascertained therefrom into the image matched to real position coordinates. Finally, such a method comprises synoptic display of the image and of the resection zone or of the joint replacement implant or vertebral replacement implant in the current position.

In a preferred development of the last-mentioned method:
a desired alignment vector of a defined body axis of the joint replacement implant or vertebral replacement implant in relation to relevant body axes in the joint region or vertebral region is determined from the integrated image,
in the step of recording the real position coordinates of the resectioning instrument or mounting tool or of the implant, an alignment vector of the current alignment thereof is determined,
any variance between the desired alignment vector and the ascertained alignment vector is calculated, and
information derived from the variance, or a control command relating to the manipulation of the resectioning instrument or mounting tool or of the implant, is output.

A step-by-step description (initially without reference to specific aspects of the arrangement) of an advantageous procedure for carrying out the method of preparing for a CT-based hip joint implantation is given below.

Basic procedure:
1. CT scan of the patient
2. calculation of 3D model from CT data
3. planning in CT slices and in the 3D model
4. measurement (determination) of the body axis coordinate system
5. transformation of the model into the body axis coordinate system
6. sensing of the surface of the acetabulum relative to the bone-fixed locator
7. calculation of the transformation parameters between 3D planning and bone-fixed adapter by minimalization of the normal spacings
8. export of the plan data into the surgical plan (coordinate system of the bone-fixed locators)
9. alignment of the calibrated instruments.

In order to be able to plan the position of the socket in three dimensions, a CT is recorded of the patient's hip. The bone structure is extracted from the individual section images, and a 3D model of the hip is calculated in which the position and alignment of the artificial socket is planned and the anatomical body axes are measured.

The position and alignment of the implant components are supplied to the further processing navigation software in the form of the desired implant position to be achieved. The plan data include the following information:
position of the body axes in the 3D model
planned center of rotation of the artificial hip joint
antetorsion angle and abduction angle of the planned socket

During the operation, first of all a bone-fixed locator is attached to the iliac crest as pelvic reference coordinate system. Access is then gained and the head of the femur is resectioned. The aim of the further procedure is to locate the model, in which the plan is known, in the actual surgical situation on the patient. For this purpose, using a manual sensor, points on the bone surface of the hip are sensed. The sensing is effected substantially in the region of the acetabulum, because here there is relatively good access to the bone surface as a result of the resectioning of the head of the femur. To a lesser extent, further points on the iliac crest are sensed on the skin.

Since the two sets of data—intra-operatively sensed points and 3D model—do not contain known identical point information, they cannot be transformed into one another directly. The scanned surface is therefore approximated in an iterative matching process on the surface of the 3D model. The intra-operatively scanned point cloud is transformed approximately into the system of body axes by way of an auxiliary beam to be measured. When so doing, the manual sensor is held approximately in the center of the acetabulum and in the direction of the planned socket implant and its position and alignment are measured.

During the subsequent matching, at each point of the sensed point cloud the normal vector from the 3D surface is calculated. The basic principle of adjustment is the minimalization of the normal spacings of all sensed points to the 3D surface with the unknowns of the spatial 3D transformation of the two coordinate systems, a constant offset and the surface inclination as weighting. The matching yields as a result the transformation parameters from the CT coordinate system to the hip-fixed coordinate system.

For matching, at each point a normal vector to the surface of the 3D model is calculated as a locally defined spatial surface. A problem is the generation of the surface for the calculation of the normal to the surface through the individual point. In conventional triangular meshing, jumps and edges cannot be avoided. This has the result that a small shift of the surface would result in extreme changes in the normal direction. This effect can be smoothed by the spatial spline approach. Unfortunately, however, in insufficiently defined regions (e.g. margin) this results in excessive vibrations which are then very far from the true surface. Therefore the definition of the unknowns of the spline parameters has been introduced, so that poorly defined regions can be ignored for the calculation. At these sites the surface is then generated by triangular meshing. The spline approach fails especially in the case of smooth surfaces, where, however, triangular meshing gives good results.

In addition, for matching a constant offset is included in the calculation. As a rule, a manual sensor having a spherical probe is used for measurements of the surface points, so that even when surfaces are actually strictly alike the sensed surface is measured shifted by the radius of the spherical probe. It has therefore proved advisable to introduce a weighting for the normal vector. Depending upon the position of the normal vector it receives a higher weighting on the basis of the quality of the unknowns of the spline facet and the actual inclination of the vector relative to the surface.

The matching enables the plan data to be transformed into the bone-fixed system, so that the instruments can be aligned in accordance with the plan data. For this purpose, the instruments need to be calibrated in accordance with the parameters and the choice of implant. The position of an instrument is measured in the hip-fixed coordinate system and transformed into the coordinate system of the body axes with the aid of the transformation parameters resulting from the matching. By calibrating the instruments, the variance between the actual position and the planned position can be displayed; the actual position can then be displayed on-line in the plan intra-operatively and the planned position can be modified in the navigation.

The procedure for the navigation of the stem can take place analogously to CT-based socket navigation.
1. data from the CT scan of the patient
2. calculation of 3D model from CT data
3. planning in CT slices and in the 3D model
4. transformation of the model into the body axis coordinate system
5. export of the plan data into the navigation
6. sensing of the surface of the femur relative to the bone-fixed femur locator intra-operatively
7. calculation of the transformation parameters by minimalization of the normal spacings
8. alignment of the calibrated instruments.

### BRIEF DESCRIPTION OF DRAWINGS

Advantages and useful features will otherwise be found in the following description of a preferred embodiment—an arrangement in connection with a method for the implantation of an artificial hip joint—in conjunction with the Figures, in which:

FIG. 1 shows a perspective view of an iliac crest locator having an associated clamp (adapter) clamped onto an iliac crest;

FIG. 2 additionally shows a perspective view of a manual sensor for sensing the table surface for the purpose of determining the table plane as well as bony references on the iliac crest (though the skin);

FIG. 3 shows, in addition to the iliac crest locator, a perspective view of a femur locator having an associated clamp for fixation in the proximal region of a femur;

FIG. 4 shows a perspective view of a sphere adapter/manual sensor combination for determining the center of the acetabulum;

FIG. 5 shows a perspective view of a milling tool/locator combination for milling the seat for a hip socket;

FIG. 6 is a diagrammatic detail view of the display of a PC monitor for visually displaying views of the milling tool relative to the pelvis;

FIG. 7 is a perspective view of a setting instrument/locator combination for screwing an artificial hip socket into the prepared seat, and

FIG. 8 is a perspective view of a medullary canal awl/locator combination for determining the path of the medullary canal in a femur.

### DETAILED DESCRIPTION OF THE PREFERRED EMBODIMENTS

The following description is given primarily with reference to a procedure for determining the relevant geometric parameters and for implanting a hip socket, but reference is additionally made also to the determination (relatively independent thereof) of the relevant geometric parameters and the implantation of a stem component as the second component of an artificial hip joint.

The operating surgeon, when planning a hip joint implantation, needs to determine the following values for the socket:

1. Size of the Artificial Socket

2. Angle of Inclination and Antetorsion Angle

The two angles of alignment of the socket axis relative to the body planes are here selected on an X-ray image by the operating surgeon in accordance with medical standpoints. These angles can likewise be modified by the operating surgeon intra-operatively.

3. Angle in the Sagittal Body Plane Between Vertical Axis and the Direction from the Iliac Crest to the Symphisis.

Determining this angle allows intra-operative determination of the body axes and thus of the plan coordinate system.

It is assumed that the patient is supine at the beginning of the operation; the physician has an X-ray image available which gives an adequate picture of the overall anatomical situation and the nature of the bones and from which he makes his first deductions as to the size of implant to be installed and the preferred approximate alignment of the implant. An incision, 4 cm in length, is made 3-5 cm dorsally of the spina iliaca superior anterior, the iliac crest is exposed and the tissue is exposed with a rasp.

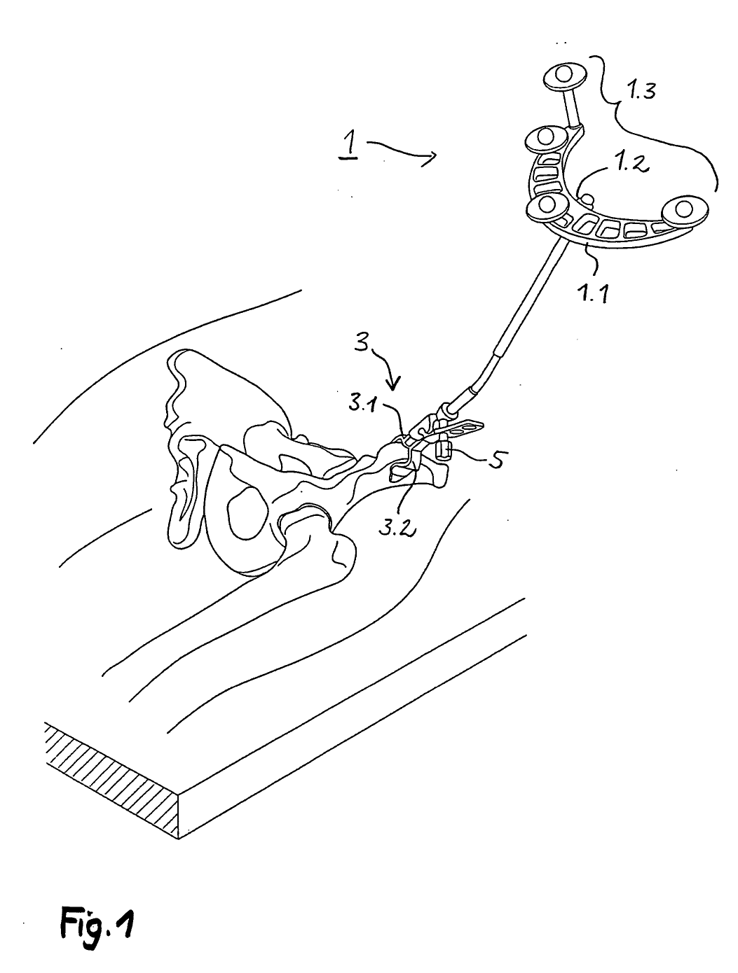

FIG. 1 shows an iliac crest locator 1 with an associated mounting clamp 3, which is attached in the exposed region of the iliac crest. The mounting clamp 3 comprises a medial clamp component 3.1 and a lateral clamp component 3.2, which are screwed together by means of an Allen bolt 5 until the mounting clamp is firmly seated on the iliac crest. The actual iliac crest locator 1 has a sickle-shaped basic body 1.1 having a mounting sleeve 1.2 for positioning on the mounting clamp 3 as well as a 4-point locator array 1.3 consisting of four IR-reflecting spheres each of which is partially surrounded by a diffuser (not separately referenced) in the shape of a spherical segment in order to avoid troublesome radiation effects. These are so-called passive targets or adapters which are known per se and the mode of operation of which in conjunction with the (likewise known) stereocamera arrangement of a so-called navigation system will therefore not be described in greater detail here. After being put in position, the locator 1 is rotated relative to the mounting clamp 3 so that the locator array is suitably aligned relative to the camera but without any of the reflecting spheres being masked by another one. Then, by screwing the locator and the mounting clamp together, a rigid connection is established between the two.

Instead of being attached to the iliac crest, the multipoint transducer 1, referred to as the iliac crest locator above, can also be attached to the roof of the aceta-bulum of the pelvis. This has the advantage that the above-mentioned (additional) incision in the region of the iliac crest becomes superfluous, but the attachment of the multipoint transducer, which is then referred to as the “surgical field locator”, is less stable if the bone structure is weak.

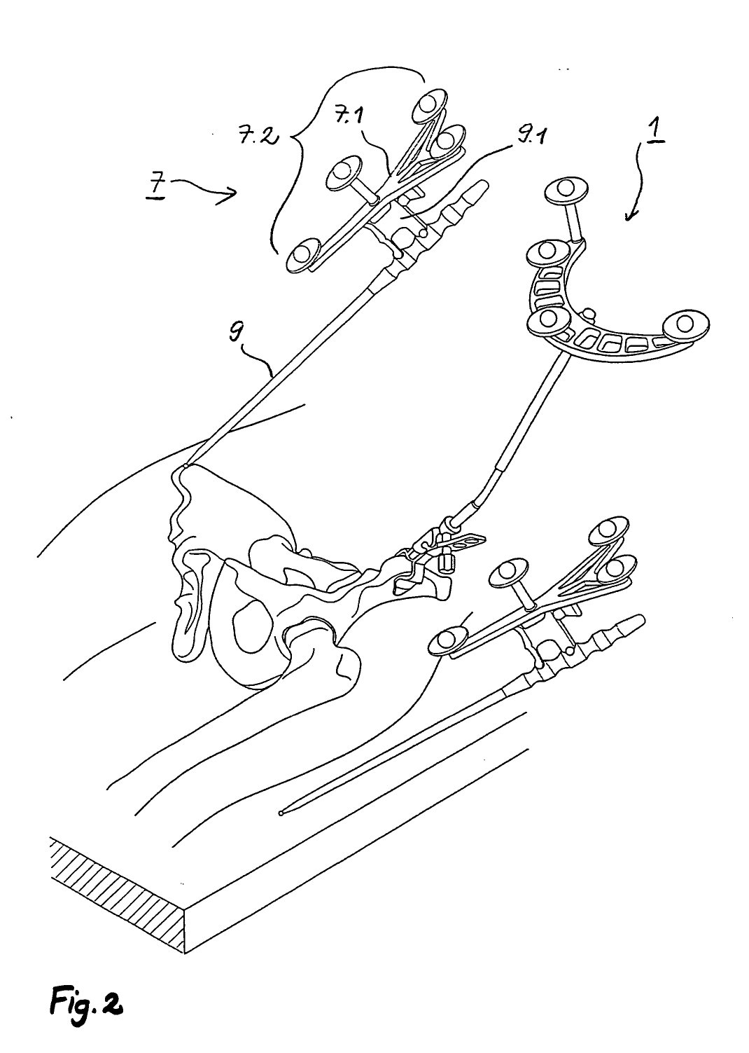

FIG. 2 shows, in addition to the above-described bone-fixed locator 1, a manual sensor 7 having a rod-shaped sensing component 9, which tapers towards one end and from which a holder 9.1 projects perpendicularly, an approximately Y-shaped sensor body 7.1 and a 4-point locator array 7.2, similar to the structure of the iliac crest locator described above. The locators of the components of the arrangement described below are also of similar structure, so that the naming of the corresponding parts and portions of those locators and the description thereof will be omitted.

Using the manual sensor 7, at the beginning of the navigation sequence various points on the plane of the operating table on which the patient is lying are scanned in order to determine the position of the table plane in space. Although this is not required for the actual determination of the patient's position, it can be used for plausibility considerations (for example in respect of the significance of the inclination of the patient's pelvis relative to the plane of the table etc.). For the actual navigation it is usually assumed that the patient's frontal plane lies parallel to the plane of the table.

Then, using the manual sensor 7, characteristic bony references in the pelvis region are sensed through the skin. First of all, the left and right iliac crests and the center of the symphysis are sensed. These three sensed points and the crest/symphysis angle ascertained during the planning enable the body axes to be clearly determined. The direction from left iliac crest to right iliac crest represents the transversal body axis. The direction from the center of the iliac crest points to the symphysis is rotated through the crest/symphysis angle about the transversal axis and thus represents the vertical body axis (orthogonal to the transversal axis). The sagittal body axis is obtained from the two first-mentioned axes as an orthogonal.

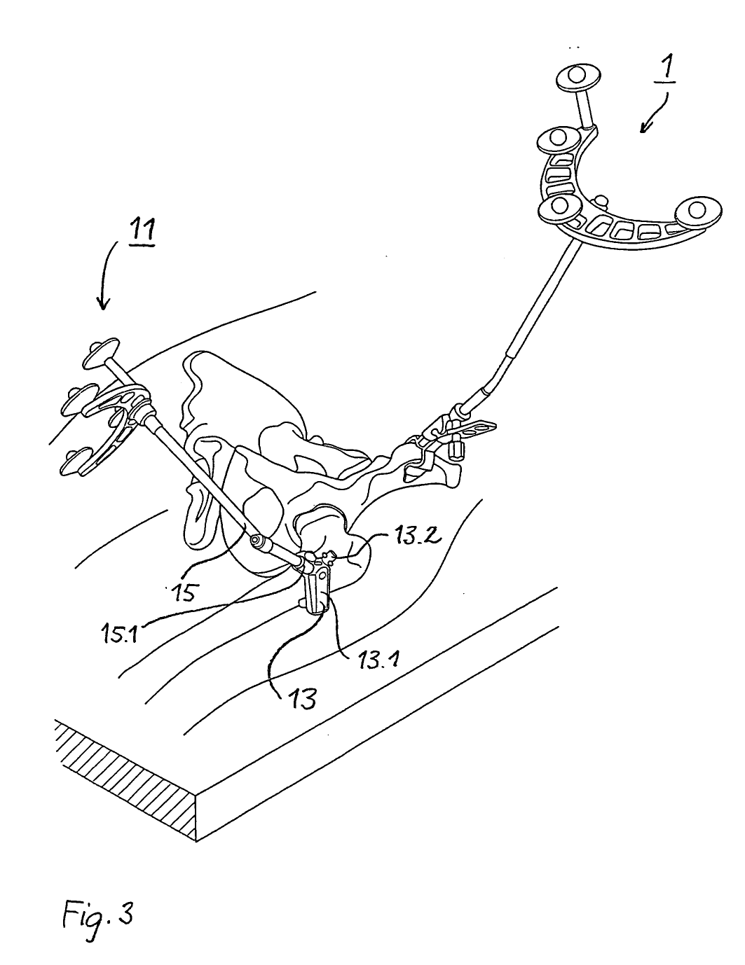

FIG. 3 shows, in addition to the iliac crest locator 1, a femur locator 11 having an associated adapter (femoral clamp) 13 for attachment close to the proximal end of the femur. The femoral clamp 13 has a two-part body consisting of a first base member 13.1, which is fork-shaped in plan view and approximately L-shaped in side view, from which two pins 13.2 project for mounting the locator, and a second base member, which is approximately L-shaped in side view and which can be locked together with the first base member 13.1. The structure of the femur locator 11 itself, apart from having an angled locator rod, is substantially the same as that of the iliac crest locator.

It is pushed by way of a mounting sleeve 15.1 at the free end of a locator rod 15 onto one of the two pins 13.2 of the femoral clamp 13.

The femoral clamp 13 is then attached to the mounted locator rod 15 on the lateral femur side approximately at the level of the trochanter minor or between the trochanter minor and the trochanter major, by pushing the muscle groups located there aside and inserting the clamp. The rotated position is to be so selected that the locator rod projects laterally out of the surgical field, if possible in the direction of the camera. Then the clamp is tightened with a moderate torque, the actual locator array (not separately referenced here) is mounted and aligned towards the camera and finally the femur locator is screwed tight.

The kinematic center of rotation of the hip is then determined both in the hip-fixed coordinate system and in the femur-fixed coordinate system by a plurality of relative measurements of the femur locator in the hip-fixed coordinate system with the leg in different positions. The transformation of all measured values can accordingly be effected from the hip-fixed coordinate system into the coordinate system of the body axes. Accordingly all the calibrated tools can then be aligned relative to the body axis coordinate system; in this connection see below. Using the center of rotation as origin, the implant can be installed at its kinematic origin. Should corrections be necessary, displacements and changes of angle in the plan can be carried out intra-operatively.

Once the operating surgeon has carried out the position recordings in the various positions of the leg in “dialogue” with the interactive user guidance (error correction again being provided on the basis of plausibility calculations), the femur locator is removed from the clamp 13 and the head of the femur is resectioned. The diameter of the resectioned head is measured and, on the basis of the measurement result, a suitable hemisphere is selected for the next step, namely the determination of the center of the acetabulum or geometric center of rotation of the hip.

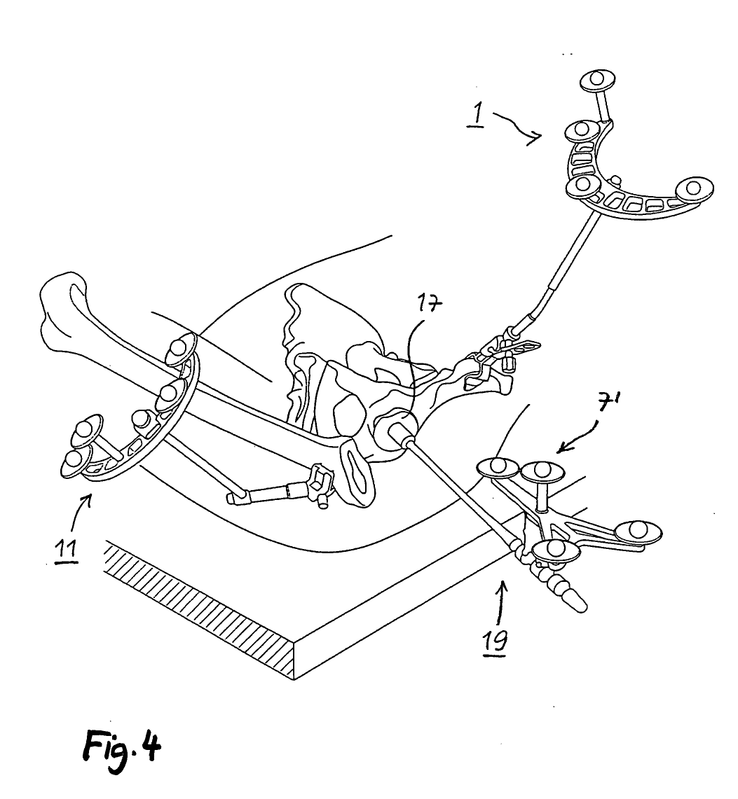

As shown in FIG. 4, the selected hemisphere 17 is combined with a manual sensor 7′ of the kind shown in FIG. 2 and described above to form a sphere adapter/manual sensor combination 19. By guiding such a locator into the socket region (usually assuming a certain anteversion angle, e.g. 12°), first the validity of the (kinematic) center of rotation determined by means of the femur locator is checked from the geometric point of view and secondly the results allow a “cross-check” of the planned implantation values from geometric standpoints. Furthermore, moving the hemisphere 17 in the socket region provides pointers to possible mechanical collisions. The structure of the half-shell and its adaptation to the manual sensor ensures that the probe tip is always in the sphere center of the sensing hemisphere.

There then follows, within the framework of the stored evaluation program with interactive user guidance, the final planning of the implantation, from the determination of the implant size that is to be installed through to displacement values and angle sizes. On that basis and with reference to previously entered specific instrument data, the system calculates desired positions for the resectioning and setting instruments to be used or, more specifically, for their operational parts.

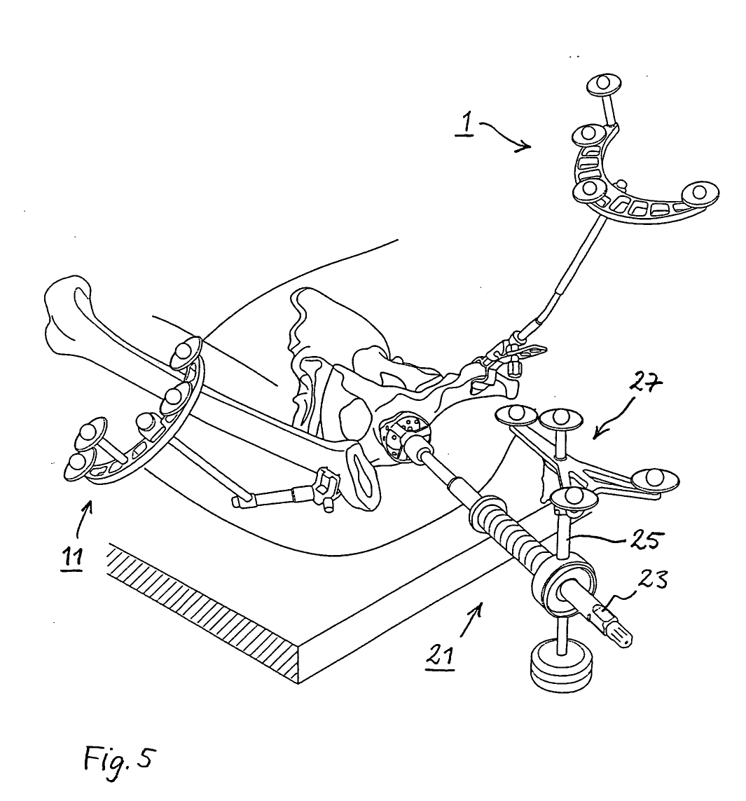

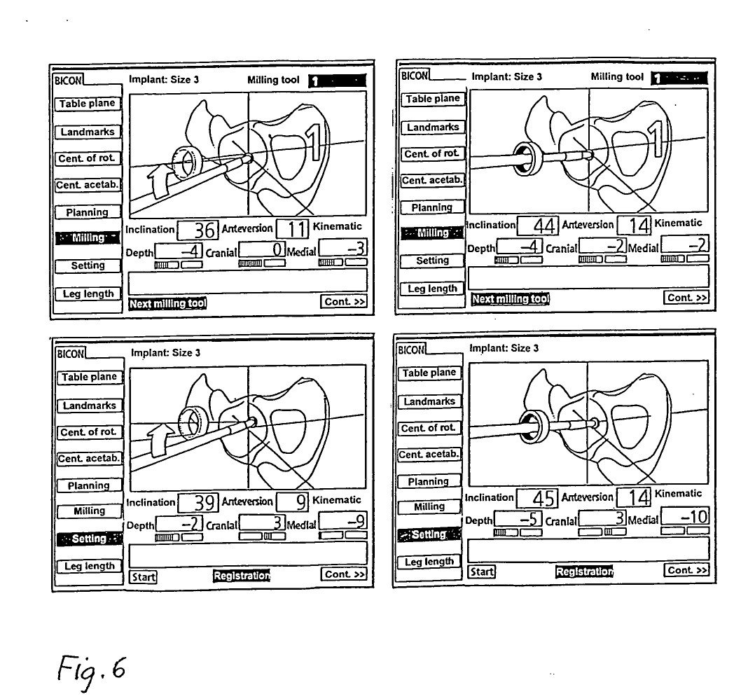

FIG. 5 shows, in addition to the iliac crest and femur locators 1, 11, a milling tool/locator combination 21 having a milling shaft 23, a milling shaft adapter 25 and a locator 27, the structure of which corresponds substantially to that of the femur locator 11 according to FIG. 3. This instrument is aligned in a socket region in the manner likewise shown in the Figure, the position and alignment being recorded on the basis of position signals from the locator array and being displayed visually on screens in the manner shown in FIG. 6. A milling tool position that is correct in accordance with the plan data is indicated on the display by a ring encompassing the milling shaft and by acoustic signals.

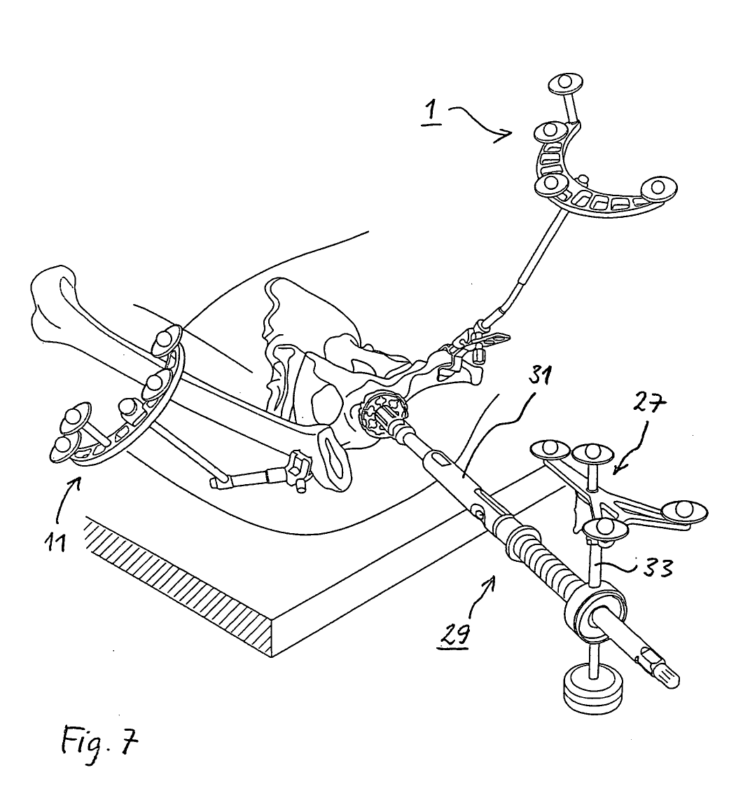

As soon as a socket seat has been produced in accordance with the plan data, the milling tool/locator combination is converted into a setting instrument/locator combination 29, as shown in FIG. 7, the locator 27 again being used but this time in conjunction with a setting instrument shaft 31 and a shaft adapter 33. Using this instrument, a hip socket 35 is set in place in a manner that is largely analogous to the manipulation of the milling tool/locator combination and that is likewise displayed on the PC screen. The ultimate position of the hip socket 35 is still to be entered into the system by the operating surgeon.

Then the stem preparation and implantation (in the first instance a test stem) are carried out, either in a conventional way or again assisted by the navigation system. Height and anteversion of the stem are fixed with reference to the plan data; only the ball neck length is still freely selectable. The joint is then assembled with the test stem, and stability and any potential for collisions during movement of the stem in the socket are tested. In addition, the leg length is roughly tested by comparing the position of the malleoli on the leg undergoing surgery and the healthy leg. If joint stability problems arise, a solution is sought by selecting a specific ball or a stem of a different size from an available range.

Optionally, in this phase it is also possible to take measurements of the other leg using the navigation system, the results of which can be used in the sense of symmetry considerations with a view to fine adjustment of the implant. It will be understood that for such measurements, instead of using the femur locator described above, there is used a femur locator modified for external mounting over the skin.

A considerable advantage of the proposed system is that using navigation data it is also possible to make a “before and after” comparison of the leg lengths (on the diseased hip prior to the operation and during the above-mentioned testing step in the final phase of the operation). For this purpose, the femur locator is again positioned and fixed in place on the holder which has remained on the femur and the position with the leg extended and aligned parallel to the longitudinal axis of the body is recorded. The position data obtained indicate any lengthening or shortening of the leg and also the so-called lateralization or medialization, that is to say the “sided” position of the femur. Where too much metallization (displacement towards the inside) is indicated, a stem different from the test stem can be used in conjuncttion with a different ball; in any case, however, the measured values suggest to the physician what should be taken into consideration in the further care of the patient.

The following remarks relate to the use of the described system in stem preparation and implantation.

The placement of the stem of a prosthetic hip requires the establishment of a planned antetorsion angle of the femur neck and the creation of the angle of the original leg length. The axial alignment of the stem is governed to a very great extent by the position of the medullary canal in the femur. As a result, it is only therefrom that the actual stem size or its offsets can be calculated.

A calibrated awl is used to determine the medullary canal of the femur. A further important item of information for the placement of the stem is the determination of the center of rotation; see above in this connection.

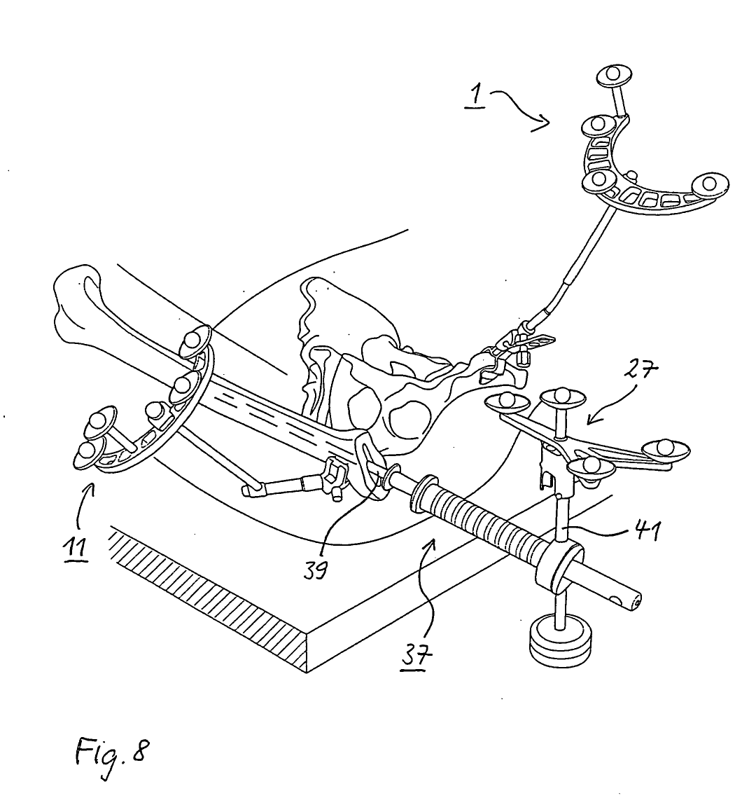

FIG. 8 shows a further component of the proposed arrangement that is suitable for use in this connection, namely a medullary canal awl/locator combination 37 having a medullary canal awl 39, an awl adapter 41 and (again) a locator 27, similar to the locator variant already shown in FIG. 3. For the insertion of this navigation instrument, the proximal femur end is opened with a box chisel or a piercing saw in the vicinity of the trochanter major and the medullary canal awl 39 is inserted therein from the proximal end.

The angle of inclination and antetorsion angle of the head of the femur are determined pre-operatively from an X-ray image and are entered intra-operatively. In addition, the antetorsion angle can be determined intra-operatively by measuring landmarks on the knee joint and on the ankle joint, so that the body planes are known intra-operatively. The actual implantation angles and positions of the socket navigation can also be taken into account in the stem implantation. The last spatial position of the socket can be applied as a relative correction of the stem. This procedure ensures optimum implantation.

The preparation of the femur for installation of the stem is then effected—analogously to the preparation of the socket seat with a navigated milling tool—with a navigated stem rasp, that is to say a stem rasp/locator combination, which is very similar to the combination shown in FIG. 8 and is therefore neither shown nor described in greater detail here. After the preparation, a test stem is again inserted and the tests described above in connection with the socket-side navigation are carried out. When satisfactory results have been obtained, the final stem is then installed without it having to be navigated again.

The invention is not limited to the arrangement described above and the procedure outlined in connection therewith, but can also be realized in modifications that lie within the scope of technical action.

### List of Reference Numerals

1 iliac crest locator
1.1 basic body
1.2 mounting sleeve
1.3 4-point locator array
3 mounting clamp
3.1 medial clamp component
3.2 lateral clamp component
5 Allen bolt
7;7′ manual sensor
7.1 sensor body
7.2 4-point locator array
9 sensing component
9.1 holder
11 femur locator
13 femoral clamp
13.1 first base member
13.2 pin
13.3 second base member
15 locator rod
15.1 mounting sleeve
17 hemisphere
19 sphere adapter/manual sensor combination
21 milling tool/locator combination
23 milling shaft
25 milling shaft adapter
27 locator
29 setting instrument/locator combination
31 setting instrument shaft
33 shaft adapter
35 hip socket
37 medullary canal awl/locator combination
39 medullary canal awl
41 awl adapter

1. An arrangement for the intra-operative determination of the spatial position and angular position of an implant selected from the group consisting of a joint replacement implant, a hip socket replacement implant, a shoulder socket replacement implant, an associated stem implant, a vertebral replacement implant, a lumbar vertebral implant, and a cervical vertebral implant, using a computer tomography method, said arrangement comprising:
a computer tomography modeling device operative to generate and store a three-dimensional image of a joint region or vertebral region to be provided with the joint replacement implant,
an optical coordinate-measuring arrangement operative to provide real position coordinates of defined real or virtual points of the joint region or vertebral region and/or position reference vectors between such points within the joint region or vertebral region or from those points to joint-function-relevant points on an extremity outside the joint region or vertebral region,
said coordinate-measuring arrangement comprising a stereocamera or stereocamera arrangement for the spatial recording of transducer signals, at least one multipoint transducer, which comprises a group of measurement points rigidly connected to one another, and an evaluation unit operative to evaluate sets of measurement point coordinates supplied by the at least one multipoint transducer and recorded by the stereocamera; and
a matching-processing unit operative to provide real position matching of the image to the actual current spatial position of the joint region or vertebral region with reference to the real position coordinates of the defined points, the matching-processing unit being configured for calculating transformation parameters with minimalization of the normal spacings.

2. The arrangement as set forth in claim 1, wherein the matching-processing unit is configured to carry out an interactive adjustment procedure for matching a sensed bone surface to a corresponding virtual surface of the image with the combined application of the principle of triangular meshing and a spatial spline approach with the definition of unknowns as spline parameters.

3. The arrangement as set forth in claim 1, further comprising an interface selected from the group consisting of an input interface operative to enter implant parameters of a predetermined set of suitable implants and to specify possible implant positions and alignments in relation to the image, and an interactive user interface having means for user guidance operative to enter implant parameters of a predetermined set of suitable implants and to specify possible implant positions and alignments in relation to the image, said interface being operatively connected to said computer tomography modeling device; wherein the matching-processing unit is connected to the input interface and is configured to determine desired coordinates or a desired movement vector of the implant being installed and of a resection area or resectioning instrument therefor comprising at least one set of entered implantation parameters, positions and alignments.

4. The arrangement as set forth in claim 3, wherein the input interface is configured for the inputting and image integration of data selected from the group consisting of the relevant body axis vectors and the implant parameters of a hip socket, and the coordinates of the center of rotation as well as the anteversion angle and the abduction angle.

5. The arrangement as set forth in claim 1, wherein a first multipoint transducer of the coordinate-measuring arrangement is in the form of a movable hand-guided sensor to sense bony references in the joint region or vertebral region in order to determine the coordinates thereof.

6. The arrangement as set forth in claim 1, wherein a second multipoint transducer is configured for rigid attachment to a bone or vertebra in the joint region or vertebral region, respectively.

7. The arrangement as set forth in claim 1, further comprising:
an instrument selected from the group consisting of a resectioning instrument, a milling tool and a rasp, which can be rigidly connected to the second or a third multipoint transducer to form a geometrically calibrated, navigable tool/transducer unit, so that from the transducer signals of that unit there can be determined real position coordinates of an operational part selected from the group consisting of an operational part of the resectioning instrument, a milling head or a rasp part, and therefrom, as desired, real position coordinates of a resection zone produced with the resectioning instrument;
an input interface configured for entering instrument parameters of the resectioning instrument which allow its synoptic display with the image of the joint region or vertebral region obtained by the computer tomography modeling device;
a matching-processing unit configured to allocate the real position coordinates of the operational part, and the real position coordinates of the resection zone to the image of the joint region or vertebral region substantially in real time; and
an image-display unit configured for synoptic display of the operational part or resection zone in its current position with the image of the joint region or vertebral region matched to real position coordinates.

8. The arrangement as set forth in claim 1, further comprising:
a tool selected from the group consisting of a mounting tool and a screwing mounting tool, operative to be rigidly connected to the second or third multipoint transducer to form a geometrically calibrated, navigable tool/transducer unit, so that the transducer signals of that unit can be used to determine real position coordinates of a part selected from the group consisting of an operational part of the mounting tool, and a screwdriver blade forming an operational part of the mounting tool, and of the implant itself;
an input interface configured to enter tool parameters of the mounting tool which allow its synoptic display with the image of the joint region or vertebral region obtained by the computer tomography modeling device;
a matching-processing unit configured to allocate the real position coordinates of the operational part and, as desired, of the implant to the image of the joint region or vertebral region substantially in real time; and
an image-display unit configured for synoptic display of the operational part or implant in its real position with the image of the joint region or vertebral region matched to real position coordinates.

9. The arrangement as set forth in claim 7, wherein the resectioning instrument and/or the mounting tool is in the form of a hand-guided tool with a handgrip having an attachment portion for rigid connection to the multipoint transducer.

10. The arrangement as set forth in claim 1, further comprising:
an adapter component configured for the rigid attachment of a multipoint transducer at a location selected from the group consisting of the joint replacement implant, and the proximal end of a stem implant, in order to create a navigable implant/transducer unit, so that the transducer signals of that unit can be used to determine real position coordinates of the adapter and thus of the implant itself,
an input interface configured to enter adapter parameters which allow synoptic display of the adapter or of the implant with the image of the joint region or vertebral region obtained by the computer tomography modeling device;
a matching-processing unit configured to allocate the real position coordinates of the adapter and of the implant to the image of the joint region or vertebral region substantially in real time; and
an image-display unit configured for synoptic display of the adapter or implant in its real position with the image of the joint region or vertebral region matched to real position coordinates.

11. The arrangement as set forth in claim 1, wherein the at least one multipoint transducer is in the form of a passive four-point transducer having four spherical reflector parts and the stereocamera or a stereocamera is in spatially fixed association with an illuminating device for illuminating the at least one multipoint transducer.

12. The arrangement as set forth in claim 2, wherein the user interface has means for providing menu guidance at least for the alignment of the three-dimensional image in relation to the relevant body axes.

13. The arrangement as set forth in claim 12, wherein the user interface has a multi-region memory operative to store the implant parameters of suitable implants or a data bank interface to an implant parameter data bank, and the means for providing menu guidance are configured for carrying out an interactive process of selecting a component with repeated access to the multi-region memory or to the implant parameter data bank.

14. The arrangement as set forth in any one of claim 3, wherein a control signal generation unit is connected to the evaluation unit and to the matching-processing unit and is configured to compare a set of implant position data or alignment data that has been entered by means of the input interface and matched to the real position coordinates of the joint region or vertebral region with currently acquired real position coordinates of the operational part of the resectioning instrument or mounting tool or implant and to determine any variance between desired position and actual position coordinates and to output variance data or a control command derived from the variance.

15. The arrangement as set forth in claim 14, wherein the control signal generation unit is configured to output at least one of a text output, a speech output and a synoptic display with the image.

16. A method for the intra-operative determination of the spatial position and angular position of an implant selected from the group consisting of a joint replacement implant, a hip socket, a shoulder socket, an associated stem implant, a vertebral replacement implant, a lumbar vertebral implant, and a cervical vertebral implant, using a computer tomography method, said method comprising the following steps:
recording a computer tomogram of the joint region or vertebral region;
processing the computer tomogram to generate a three-dimensional image of the joint region or vertebral region and storing that image as a plan model;
determining relevant body axes in the plan model and allocating those axes to a body axis coordinate system;
obtaining real position coordinates of defined real or virtual points of the joint region or vertebral region and/or position reference vectors between such points within the joint region or from those points to joint-function-relevant points on an extremity outside the joint region by means of a navigation procedure using a stereocamera or stereocamera system, at least one multipoint transducer having a group of measurement points rigidly connected to one another, and an evaluation unit for evaluating sets of measurement point coordinates supplied by the multipoint transducer and recorded by the stereocamera; and
carrying out matching-processing for real position matching of the image to the actual current spatial position of the joint region or vertebral region with reference to real position coordinates of the defined points, the matching-processing unit being configured for calculating transformation parameters with minimalization of the normal spacings.

17. The method as set forth in claim 16, further comprising:
entering implantation parameters of a predetermined set of suitable joint replacement implants or vertebral replacement implants and image-related desired coordinates for specifying possible implant positions and alignments thereof; and
integrating an image of the joint replacement implant or vertebral replacement implant into the image and displaying a synoptic representation from the images prior to the matching-processing step.

18. The method as set forth in claim 17, wherein the input and display steps are carried out, with multiple repetitions, in the context of interactive user guidance through to a final determination of the selected joint replacement implant or vertebral replacement implant and of a defined implant position and alignment and its display with the three-dimensional image.

19. The method as set forth in claim 16, further comprising:
recording the real position coordinates of an operational part of a resectioning instrument or mounting tool or of a joint replacement implant or vertebral replacement implant rigidly connected to a multipoint transducer by means of a coordinate-measuring arrangement;
integrating the real position coordinates of a resection zone or the joint replacement implant or vertebral replacement implant ascertained therefrom into the image matched to real position coordinates; and
synoptically displaying the image and the resection zone or the joint replacement implant or vertebral replacement implant in the current position.

20. The method as set forth in claim 19, wherein:
a desired alignment vector of a defined body axis of the joint replacement implant or vertebral replacement implant in relation to relevant body axes in the joint region or vertebral region is determined from the integrated image;
in the step of recording the real position coordinates of the resectioning instrument or mounting tool or of the implant, an alignment vector of the current alignment thereof is determined,
any variance between the desired alignment vector and the ascertained alignment vector is calculated, and
information derived from the variance, or a control command relating to the manipulation of the resectioning instrument or mounting tool or of the implant, is output.
